(19) United States
(12) Patent Application Publication
(10) Pub. No.: US 2010/0249439 A1
(43) Pub. Date: Sep. 30, 2010

(54) PROCESS FOR CONTROLLED CRYSTAL SIZE IN 1,2-SUBSTITUTED 3,4-DIOXO-1-CYCLOBUTENE COMPOUNDS

(76) Inventors: Vincenzo Liotta, Glen Ridge, NJ (US); Xu Yanke, Warren, NJ (US)

Correspondence Address: MERCK;PATENT DEPARTMENT (K-6-1, 1990), 2000 GALLOPING HILL ROAD, KENILWORTH, NJ, 07033-0530, US

(21) Appl. No.: 12/665,290

(22) PCT Filed: Jul. 1, 2008
(86) PCT No.: PCT/US08/08188

§ 371 Date: Jun. 11, 2010

Related U.S. Application Data
(60) Provisional application No. 60/958,636, filed on Jul. 5, 2007.

(51) Int. Cl. C07D 307/54

(52) U.S. Cl. 549/494

(57) ABSTRACT

This application discloses a novel process for the preparation of 2-Hydroxy-N,N-dimethyl-3-[[2-[[1(R)-(5-methyl-2-furanyl)propyl]amino]-3,4-dioxo-1-cyclobuten-1-yl]amino]benzamide, which has utility, for example, in the treatment of CXC chemokine-mediated diseases.

### CROSS REFERENCE TO RELATED APPLICATIONS

This application is based on and claims the priority of U.S. Provisional Patent Application No. 60/958,636, filed Jul. 5, 2007, the description of which is incorporated herein by reference in its entirety.

### FIELD OF THE INVENTION

This application discloses a novel process for the preparation of 1,2-substituted 3,4-dioxo-1-cyclobutene compounds, which have utility, for example, in the treatment of CXC chemokine-mediated diseases, and intermediates useful in the synthesis thereof.

### BACKGROUND OF THE INVENTION

Identification of any publication, patent, or patent application in this section or any section of this application is not an admission that such publication is prior art to the present invention.

The preparation of 1,2-substituted 3,4-dioxo-1-cyclobutene compounds, for example, 2-Hydroxy-N,N-dimethyl-3-[[2-[[1(R)-(5-methyl-2-furanyl)propyl]amino]-3,4-dioxo-1-cyclobuten-1-yl]amino]benzamide (compound of formula I):

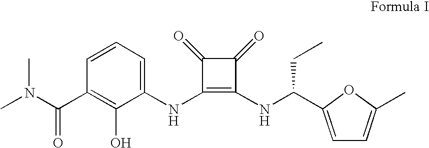

has been described in U.S. Pat. Nos. 7,123,445 (the '445 patent), issued Nov. 7, 2006, and 7,071,342 (the '342 patent), issued Jul. 4, 2006, the disclosure of each of which is incorporated herein in its entirety by reference. For examples of the preparation of the compound of Formula I, see the '455 patent at cols. 491 to 492, cols. 196 to 197, and cols. 251 to 256, and see the '342 patent, for example, at cols. 22 through 24.

Another example of the preparation of a 1,2-substituted 3,4-dioxo-1-cyclobutene compound, the preparation the 2-hydroxy-N,N-dimethyl-3-[[2-[[1(R)-[5-methyl-4-(1-methylethyl)-2-furanyl]propyl]amino]-3,4-dioxo-1-cyclobuten-1-yl]amino]-benzamide (the compound of Formula II)

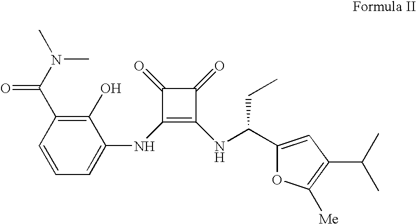

is described in U.S. provisional patent application 60/819,541 (the '541 application) filed Jul. 7, 2006, the disclosure of which is incorporated by reference in its entirety. An example of the preparation of the compound of Formula II can be found in Example 2 of the '541 application. The aforementioned preparation schemes for the compounds of Formulae I and II are incorporated herein by reference in their entirety.

The synthesis method for preparing 1,2-substituted 3,4-dioxo-1-cyclobutene compounds described in the '342 patent generally follows Scheme I (which exemplifies the preparation of 2-Hydroxy-N,N-dimethyl-3-[[2-[[1(R)-(5-methyl-2-furanyl)propyl]amino]-3,4-dioxo-1-cyclobuten-1-yl]amino]benzamide, the compound of Formula I).

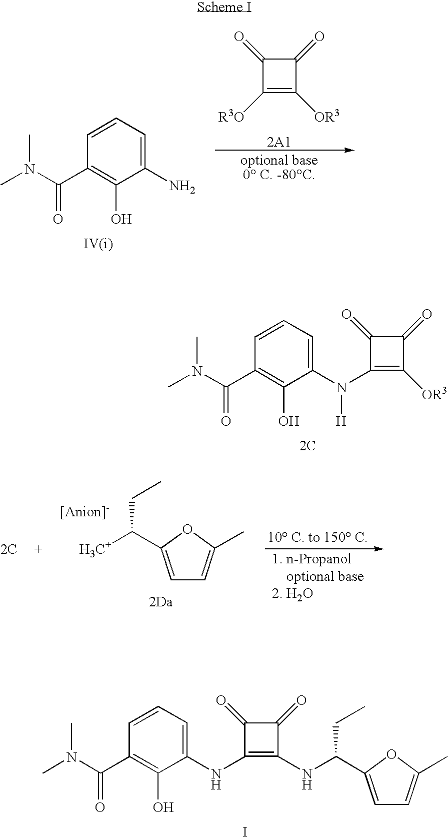

The process for the preparation of the compound of Formula I shown in Scheme I is carried out by first preparing intermediate compound 2C from a dialkyl squarate, a strong skin sensitizer and irritant which is difficult to handle. Additionally, the conditions described in the aforementioned publications under which compounds 2C and 2Da are coupled in the second step of Scheme I produce an undesirable level of impurities admixed with the final product.

### BRIEF DESCRIPTION OF THE FIGURES

FIG. 1 presents a characteristic x-ray powder diffraction pattern of the crystalline Form IV of the compound of Formula I [Vertical Axis: Intensity CPS, counts (square root)); Horizontal Axis: Two Theta (degrees)].

### OBJECTIVES AND SUMMARY OF THE INVENTION

In view of the foregoing, what is needed is a method of providing crystals of the monohydrate Form 4 of the compound of Formula I which can be efficiently isolated by filtration. What is needed also is a reaction scheme which affords practical scale up to a batch size suitable for commercial scale preparation.

These and other objectives are advantageously provided by the present invention, which in one aspect is a process for preparing crystals of 2-Hydroxy-N,N-dimethyl-3-[[2-[[1(R)-(5-methyl-2-furanyl)propyl]amino]-3,4-dioxo-1-cyclobuten-1-yl]amino]benzamide (compound of formula I) affording filter cake specific resistance of less than 7.9×10¹¹ m/Kg,

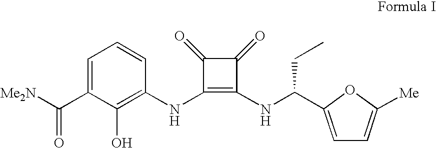

the process comprising:
(a) providing a solution of the compound of Formula I in a mixture of solvent and anti-solvent at the temperature of dissolution of the compound of Formula I afforded by the solvent/anti-solvent system selected;
(b) cooling the solution from step “a” to a temperature just above a temperature at which nucleation of the compound of Formula I commences in the solvent/antisolvent mixture selected for step “a” and seeding the batch with a solid crystalline form of the compound of Formula I monohydrate Form 4, thereby forming a mixture;
(c) cooling the mixture from step “b” using a cooling rate of from about 0.01° C./min. to about 5° C./min to a temperature at which substantially all of the compound of Formula I dissolved in step “a” is crystallized into a slurry; and
(d) cycling the temperature of the slurry from step “c” by heating it at a rate of from about 0.01° C./min, to about 5° C./min to a temperature below the temperature of seeding employed in step “b” and cooling the heated slurry at a rate of from about 0.01° C./min. to about 5° C./min to a temperature of about the crystallization temperature achieved in step “c”, and repeating the cycle until crystals of the desired cross-section are obtained, thereby providing a filter cake specific resistance of less than 7.9×10¹¹ m/Kg when the precipitated crystals are isolated by filtration.

In some embodiments of the inventive process, it is preferred to add acid to the solution prior to the first heating step “a”, preferably acetic acid.

In some embodiments it is preferred to select the solvent from alcohols having 6 carbon atoms or less, acetone, acetonitrile, tetrahydrofuran, and N-methylpyrrolidine, preferably, alcohols having 6 carbon atoms or less, more preferably, the solvent is n-propanol. In some embodiments it is preferred to use the solvent:anti-solvent in a ratio of from about 5 vol % solvent:95 vol % antisolvent to about 98 vol % solvent:2 vol % anti-solvent. In some embodiments it is preferred to use water as the antisolvent. In some embodiments using n-propanol as the solvent, it is preferred to use a 1:1 mixture of n-propanol and water. In some embodiments of the inventive process using n-propanol as the solvent, it is preferred to dissolve the compound of Formula I in step “a” at a temperature of about 70° C.

In some embodiments using n-propanol it is preferred to cool the solution in step “b” to a temperature of at most about 62° C. prior to seeding the solution.

In some embodiments using n-propanol it is preferred to use a cooling rate in step “c” of from about 0.01° C./min. to about 5° C./min., more preferably a cooling rate of 0.1° C./min, and to cool the mixture thereby to a temperature of about 20° C.

In some embodiments using n-propanol, in cycling step “d”, during heating cycles it is preferred to heat the mixture to a temperature of about 53° C. at a heating rate of about 0.5° C./min, and during cooling cycles to cool the mixture to a temperature of about 20° C./min. at a cooling rate of about 0.1° C./min, and repeat the cycling between those temperatures and at those heating and cooling rates until crystals of a desired size are produced.

In some embodiments of the inventive process it is preferred to perform 4 heating and cooling cycles is step “d”. In some embodiments of the inventive process it is preferred to perform 8 heating cycles. In some embodiments of the inventive process it is preferred to see the solution in step “b” with crystals previously prepared by the inventive process using at least 4 heating and cooling cycles in step “d”.

In some embodiments it is preferred to prepare a solution in Step “a” by admixing the isolated solid compound of Formula I with a solvent to dissolve the compound and adding an antisolvent to the resulting solution.

In some embodiments of the inventive process it is preferred to provide a solution in step “a” by adding an aliquot of n-propanol to the reaction mixture in which the compound of Formula I was prepared, concentrate the reaction mixture by distillation, adding a second aliquot of n-propanol, concentrate the mixture a second time by distillation, adding a third aliquot of n-propanol and acetic acid, filtering the reaction mixture, adding additional n-propanol and heating the mixture, then adding water, seeding the mixture with crystals of the compound of Formula I Form 4, and cooling the mixture to the desired crystallization temperature, preferably about 20° C., followed by cycling the temperature between a temperature below the seeding temperature, preferably below about 62° C. and crystallization temperature, until the desired crystal size is obtained.

Other aspects and advantages of the invention will become apparent from following Detailed Description.

### DETAILED DESCRIPTION OF THE INVENTION

Terms used in the general schemes herein, in the examples, and throughout the specification, include the following abbreviations, together with their meaning, unless defined otherwise at the point of their use hereinafter: Me (methyl); Bu (butyl); t-Bu (tertiary butyl); Et (ethyl); Ac (acetyl); t-Boc or t-BOC (t-butoxycarbonyl); DMF (dimethylformamide); THF (tetrahydrofuran); DIPEA (diisopropylethylamine); MTBE (methyltertiarybutyl ether); 2-Me-THF

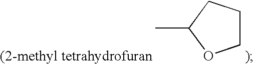

n-propyl, n-prop (CH₃CH₂CH₂—); RT (room temperature, ambient temperature, generally 25° C.); TFA (trifluoroacetic acid); TEA (triethyl amine).

As used herein, the following terms, unless otherwise indicated, are understood to have the following meanings:

The term “substituted” means that one or more hydrogens on the designated atom or group of atoms in a structure is replaced with a selection from the indicated group, provided that the designated atom's normal valency under the existing circumstances is not exceeded, and that the substitution results in a stable compound. Combinations of substituents and/or variables are indicated when such combinations result in stable compounds. By “stable compound” or “stable structure” is meant a compound that is sufficiently robust to survive isolation to a useful degree of purity from a reaction mixture, and formulation into an efficacious therapeutic agent.

The term “optionally substituted” means optional substitution with the specified groups, radicals or moieties.

“Patient” includes both humans and animals.

“Mammal” means humans and other mammalian animals.

“Alkyl” means an aliphatic hydrocarbon group which may be linear straight or branched and comprising about 1 to about 10 carbon atoms in the chain. Branched means that one or more lower alkyl groups such as methyl, ethyl or propyl, are attached to a linear alkyl chain. Non-limiting examples of suitable alkyl groups include methyl, ethyl, n-propyl, isopropyl, n-butyl, t-butyl and n-pentyl.

“Alkenyl” means an aliphatic hydrocarbon group containing at least one carbon-carbon double bond and which may be straight or branched and comprising about 2 to about 10 carbon atoms in the chain. Branched means that one or more lower alkyl groups such as methyl, ethyl or propyl, are attached to a linear alkenyl chain. Non-limiting examples of suitable alkenyl groups include ethenyl, propenyl, n-butenyl, 3-methylbut-2-enyl and n-pentenyl.

“Alkylene” means a difunctional group obtained by removal of an additional hydrogen atom from an alkyl group, as “alkyl” is defined above. Non-limiting examples of alkylene include methylene (i.e., —CH₂—), ethylene (i.e., —CH₂—CH₂—) and branched chains, for example, —CH(CH₃)—CH₂—.

“Aryl” means an aromatic monocyclic or multicyclic ring system comprising about 6 to about 14 carbon atoms, preferably about 6 to about 10 carbon atoms. The aryl group can be optionally substituted with one or more “ring system substituents” which may be the same or different, and are as defined herein. Non-limiting examples of suitable aryl groups include phenyl and naphthyl.

“Cycloalkyl” means a non-aromatic mono- or multicyclic ring system comprising about 3 to about 10 carbon atoms, preferably about 3 to about 6 carbon atoms. Non-limiting examples of suitable monocyclic cycloalkyls include cyclopropyl, cyclobutyl, cyclopentyl, cyclohexyl, cycloheptyl and the like. Non-limiting examples of multicyclic cycloalkyls include, but are not limited to 1-decalin, norbornyl and cognitors, adamantyl and cognitors.

“Halo” means a halogen selected from fluoro, chloro, bromo, or iodo groups.

“Aminoalkyl” means an alkyl as defined above having at least one hydrogen atom on the alkyl moiety replaced by an amino functional (i.e., —NH₂) group. Alkylamino means an amino functional group having one or both hydrogens replaced by an alkyl functional group, as “alkyl” is defined above.

With reference to the number of moieties (e.g., substituents, groups or rings) in a compound, unless otherwise defined, the phrases “one or more” and “at least one” mean that there can be as many moieties as chemically permitted, and the determination of the maximum number of such moieties is well within the knowledge of those skilled in the art.

A wavy line [symbol] appearing on a structure and joining a functional group to the structure in the position of a bond generally indicates a mixture of, or either of, the possible isomers, e.g., containing (R)- and (S)-stereochemistry. For example,

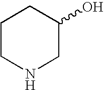

means containing either, or both of

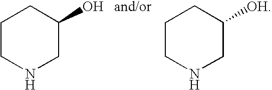

A wavy line which terminates a bond indicates that the portion of the structure depicted is attached to a larger structure at the indicated bond, for example,

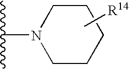

implies that the nitrogen of the substituted piperidyl group depicted is bonded to an undepicted structure on which it is a substituent.

Lines drawn into ring systems, for example the substituted aryl group:

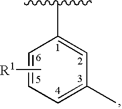

indicates that a substituent (R¹) may replace a hydrogen atom of any of the ring carbons otherwise bonded to a hydrogen atom. Thus, as illustrated, R¹ can be bonded to any of carbon atoms 2, 4, 5, or 6, but not 3, which is bonded to a methyl substituent, or 1, through which the substituted aryl group is bonded.

As well known in the art, a bond drawn from a particular atom wherein no moiety is depicted at the terminal end of the bond indicates a methyl group bound through that bond to the atom, unless stated otherwise. For example:

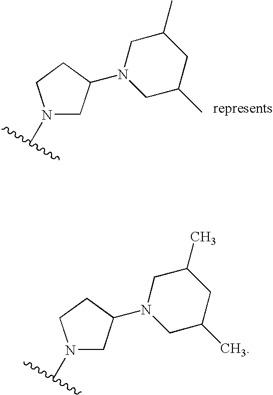

However, sometimes in the examples herein, the CH₃ moiety is explicitly included in a structure. As used herein, the use of either convention for depicting methyl groups is meant to be equivalent and the conventions are used herein interchangeably for convenience without intending to alter the meaning conventionally understood for either depiction thereby.

The term “isolated” or “in isolated form” for a compound refers to the physical state of said compound after being isolated from a process. The term “purified” or “in purified form” for a compound refers to the physical state of said compound after being obtained from a purification process or processes described herein or well known to the skilled artisan, in sufficient purity to be characterizable by standard analytical techniques described herein or well known to the skilled artisan.

When any variable (e.g., aryl, heterocycle, R², etc.) occurs more than one time in any constituent or in a formula, its definition on each occurrence is independent of its definition at every other occurrence.

As mentioned above, a process for preparing each of the compounds of Formula I and Formula II have been described U.S. Pat. No. 7,123,455 (the '455 patent, both compounds) and U.S. Pat. No. 7,071,342 (the '342 patent, the compound of Formula I). The present invention utilizes the processes depicted in Schemes IIa and IIb to prepare the compounds of Formula Ia, for example, the compounds of formulae I and II. Aspects of the preparation and purification of the compounds of Formulae I and flare also discussed in U.S. provisional application Ser. Nos. 60/956,317; 60/958,313: and 60/958,311, each of which was filed on Jul. 3, 2007, and in copending application filed internationally herewith under attorney docket number CD06674US01, the disclosure of each of which is incorporated herein by reference in its entirety.

Scheme IIa presents a coupling reaction between a salt of an amino-furate (2Da) and an amino-substituted hydroxyl-benzamide (2C) which is carried out in 2-methyl-tetrahydrofuran (2-MeTHF).

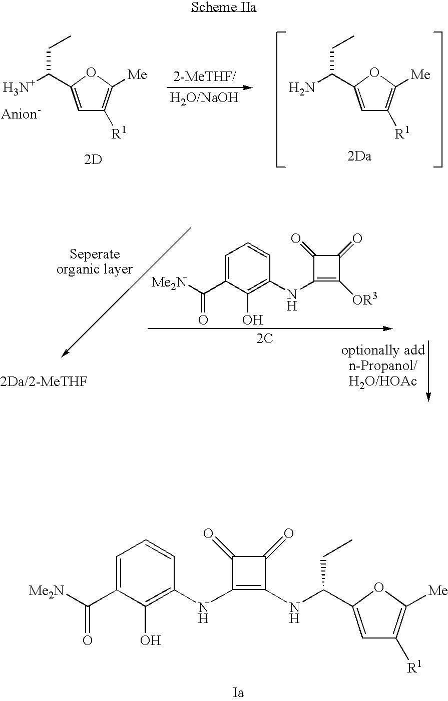

The coupling reaction depicted in Scheme IIa is a process comprising:
(a) forming a free base amino-furan compound 2Da from the amino-furan salt compound of the formula 2D wherein, R¹ is selected from hydrogen and a substituent comprising from 1 carbon atom to about 10 carbon atoms selected from linear, branched, and cyclic alkyl moieties and substituted linear, branched, and cyclic alkyl moieties and “anion” represents a monovalent anionic moiety;
(b) reacting said free base amino-furan compound 2Da with a hydroxyaminobenzamide compound of Formula 2C to provide the compound of Formula Ia; and
(c) optionally precipitating the compound of Formula Is by:
   (i) successive cycles of concentrating the reaction mixture formed in step “c” by distillation followed by the addition of an aliquot of n-propanol;
   (ii) adding an aliquot of acetic acid and n-propanol to the concentrate formed in step “i”;
   (iii) heating the solution formed in step “ii”;
   (iv) adding an aliquot of water and seed crystals comprising the compound of Formula Ia to the hot solution from step “iii”;
   (v) cycling the temperature of the seeded solution prepared in step “iv” until a slurry comprising crystals of a desired size is formed; and
   (vi) optionally isolating the crystals from the slurry prepared in step “v”.
      The inventors have surprisingly found that the coupling reaction between the hydroxyaminobenzamide and aminofuran shown in Scheme IIa (step “a”) occurs with an improved impurity profile if the coupling reaction is conducted in 2-methyltetrahydrofuran. Conveniently, the aminofuran used in the coupling reaction shown in Scheme IIa can be provided by liberating the free base form of the aminofuran to be reacted from its corresponding salt. Thus, a 2-methyltetrahydrofuran solution of the aminofuran free base is provided by treatment of a 2-methyl-tetrahydrofuran suspension of the salt with a strong aqueous base. Upon reaction with the aqueous base, the freebase form of the aminofuran is liberated and dissolves in the 2-methyltetrahydrofuran suspending solvent. The organic layer of the reaction mixture is then easily separated from the reaction mixture by physical means, for example, separation and decantation. The hydroxyaminobenzamide (2C) to be coupled with the aminofuran is added to the 2-methyltetrahydrofuran solution containing the aminofuran freebase, and heated to initiate the coupling reaction. The reaction can be carried out at temperatures above 0° C., preferably a temperature of at least 40° C., and more preferably the reaction is carried out at a temperature of about 70° C.

In some embodiments it is preferred for the limiting reagent selected to be the hydroxyaminobenzamide. In some embodiments, preferably after a substantial portion of the limiting reagent has been consumed, aliquots of n-propanol are added to the reaction mixture with subsequent distillation to reduce the volume of the reaction mixture. In some embodiments it is preferred to carry out several cycles of adding n-propanol and subsequently distilling volatiles from the reaction mixture until the reaction mixture comprises substantially n-propanol, thus facilitating the separation of the product compound of Formula Ia from the reaction mixture by crystallization. To this end a final aliquot of n-propanol and a small amount of acetic acid is added to neutralize any residual base, thereby maximizing yield. The mixture is subsequently filtered and the filtrate is diluted with additional n-propanol and heated to at least 70° C. Water is added to the heated mixture as an antisolvent while maintaining the temperature. The mixture is then cooled to about 60° C. and seed crystals of the compound of Formula Ia are added and the mixture is subjected to controlled cooling to facilitate crystallization of the compound of Formula Ia.

The inventors have found that in some embodiments, for example, when the compound of Formula Ia is the compound of Formula I, cycling the temperature of the seeded mixture between ambient temperature and a temperature of from about 50° C. to about 60° C. permits control of the size of the crystals formed.

For use in carrying out the synthesis shown in Scheme IIa, above, the aminohydroxybenzamide intermediate compounds of Formula 2C are conveniently prepared by reacting a dialkyl squarate, for example, dimethyl squarate and diethyl squarate, preferably, dimethyl squarate, and the compound of 2B in accordance with Scheme IIb, shown below.

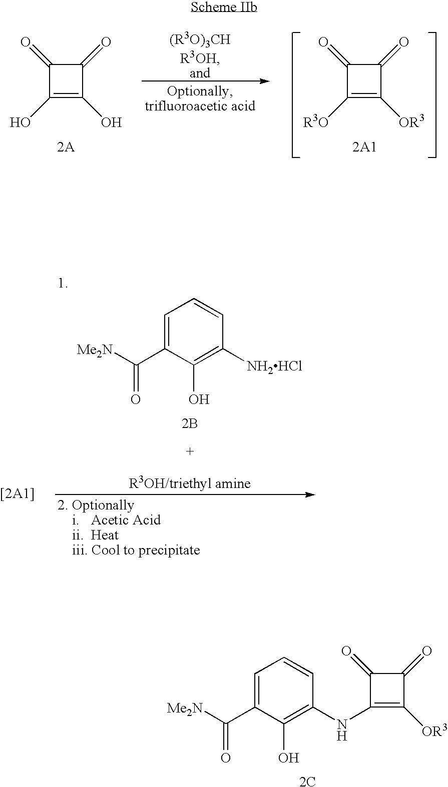

The reaction shown in Scheme IIb is a process comprising:
(a) forming dialkyl-squarate compounds of Formula 2A1 in situ by reacting (R³O)₃CH (trialkylorthoformate) with squaric acid (2A), wherein R³ is a linear or branched alkyl of 6 carbon atoms or less; and
(b) reacting the compound of Formula 2A1 prepared in step “a” with a salt of a 2-hydroxy-2-amino-benzamide compound of Formula 26.

Surprisingly, the inventors have found that the coupling reaction schematically shown in Scheme IIb can be carried out by generating the dialkyl squarate in situ from a reaction between squaric acid (compound 2A) and a trialkylorthoformate [(R³O)₃CH]. Preferably the trialkylorthoformate is selected from trimethyl orthoformate and triethylorthoformate, more preferably trimethylorthoformate. In some embodiments it is preferred to use a slight excess of trialkylorthoformate in comparison to the amount of squaric acid employed. In some embodiments it is preferred to use about 1 equivalent of squaric acid and about 2.1 equivalents of trialkylorthoformate.

Optionally, the esterification reaction is catalyzed with a small amount of acid. When an additional acid is employed, preferably the acid is trifluoroacetic acid. In some embodiments of the inventive process using trifluoroacetic acid to catalyze the reaction between trimethylorthoformate and squaric acid it is preferred to use about 1 mole % of trifluoroacetic acid relative to the amount of trimethylorthoformate employed.

Squaric acid is an article of commerce available, for example, from Aldrich. The inventors have surprisingly found that generating dialkylsquarate (2A1) in situ from squaric acid (2A) permits the process to be run without requiring isolation and handling a dialkyl squarate in the preparation of the intermediate compound (2C). Dialkylsquarates are known to be irritants and skin sensitizers. The present process, in generating the dialkylsquarate in situ for use in preparing intermediate 2C thus eliminates the necessity of handling dialkyl squarate and thereby improves the safety and scalability of the process.

Any trialkyl orthoformate of the formula [(R³O)₃CH], wherein R³ is linear or branched alkyl having 6 carbon atoms or less is suitable for carrying out step 1 of dialkylsquarate synthesis reaction shown in Scheme IIb, preferably, the reaction is carried out with a trialkylorthoformate selected from triethylorthoformate, thus the compound of Formula 2A1 is diethylsquarate, and trimethyl orthoformate, thus the compound of Formula 2A1 is dimethylsquarate, more preferably the reaction is carried out with trimethyl orthoformate. It will be appreciated that other methods of generating dialkylsquarates in situ can also be employed without departing from the scope of the present inventive reaction.

Preferably, in situ generation of dialkyl squarate is carried out in a refluxing alcohol having the structure (R³O)₃CH, in which R³— is selected to be the same as the alkyl moiety present in the trialkylorthoformate used to react with squaric acid to generate the dialkyl squarate. Thus, for example, when diethyl squarate is prepared using triethylorthoformate it is preferred to carry out the reaction in ethanol, and when dimethyl squarate is prepared using trimethylorthoformate, it is preferred to carry out the reaction in methanol. Conveniently, the alcohol selected on this basis to carry out the in situ generation of dialkyl squarate is also a suitable solvent for carrying out the preparation of the compound of Formula 2C by coupling the dialkylsquarate generated in situ and the aminohydroxybenzamide salt compound of Formula 2B in accordance with step 2 of Scheme IIb. Thus, conveniently, when dialkylsquarate is made in situ in accordance with Step 1 of Scheme IIb, the solution prepared in step 1 can be used directly in the coupling reaction of step 2.

In some embodiments, at the end of the refluxing period for preparing dialkylsquarate, it is preferred to concentrate the reaction mixture by distilling volatiles from the reaction mixture. In some embodiments using methanol as the reaction solvent, it is preferred to concentrate the solution containing the dialkylsquarate prepared in situ by refluxing the reaction mixture until it reaches a temperature of about 70° C.

After the alcohol solution of dialkylsquarate is prepared in accordance with step 1 of Scheme IIb, it can be used directly in the formation of the compound of Formula 2C shown in Step 2 of Scheme IIb. In some embodiments, after concentrating the reaction mixture, in preparation to carry out the formation of the compound of the Formula 2C, it is preferred to dilute the concentrated solution containing the dialkylsquarate to 6× the volume with aliquots of additional alcohol. In some embodiments it is preferred to carry out the coupling reaction at a temperature of less than about 30° C., more preferably at a temperature of from about [−10° C.] to about [+10° C.], and more preferably at a temperature of from about [−5° C.] to about [+5° C.].

In some embodiments, after cooling the solution of dialkyl squarate, the amino-hydroxybenzamide salt of formula 2E3 is added to the alcoholic solution of dialkylsquarate in an amount that provides from about 0.5 equivalents to about 1.0 equivalents of the benzamide salt in comparison with the dialkylsquarate employed, preferably about 0.7 equivalent of the benzamide salt is employed. In some embodiments it is preferred to mediate the coupling reaction with an organic base, for example, but not limited to pyridine, pyridine derivatives, and tertiary amines, for example, but not limited to, triethyl amine. Preferably the base is a tertiary amine, more preferably it is selected from diisopropylethylamine and triethyl amine, more preferably the base is triethylamine. When used, it is preferred to employ at least about one equivalents of the base in comparison with the amount of benzamide salt employed, preferably about 1.8 equivalents.

In some embodiments using triethylamine to mediate the coupling reaction, it is preferred to add the triethylamine over a period of the reaction time, preferably about two thirds of the reaction period, while maintaining the reaction mixture temperature from about [−5° C.] to about [+5° C.]. In some embodiments utilizing triethylamine, it is preferred to work up the reaction after the reaction period by seeding the reaction mixture with the solid amounts of the compound of Formula 2C to nucleate crystal growth, then add acetic acid to insure that any base still present is neutralized, thus maximizing yields of the coupled product. When used, it is preferable to add an amount of acetic acid equivalent to twice the mole amount of triethylamine added. In some embodiments employing acetic acid, following acid addition it is preferred to heat the reaction mixture, preferably to at least 60° C., more preferably to a temperature of from about 60° C. to about 70° C., then reduce the temperature in controlled stages, preferably, first to a temperature of less than about 35° C., more preferably to a temperature of from about 25° C. to about 35° C., followed by a period of time in which the reaction mixture is cooled, preferably to a temperature of from about [−5° C.] to about [−5° C.], to precipitate crystals of the intermediate compound of Formula 2C.

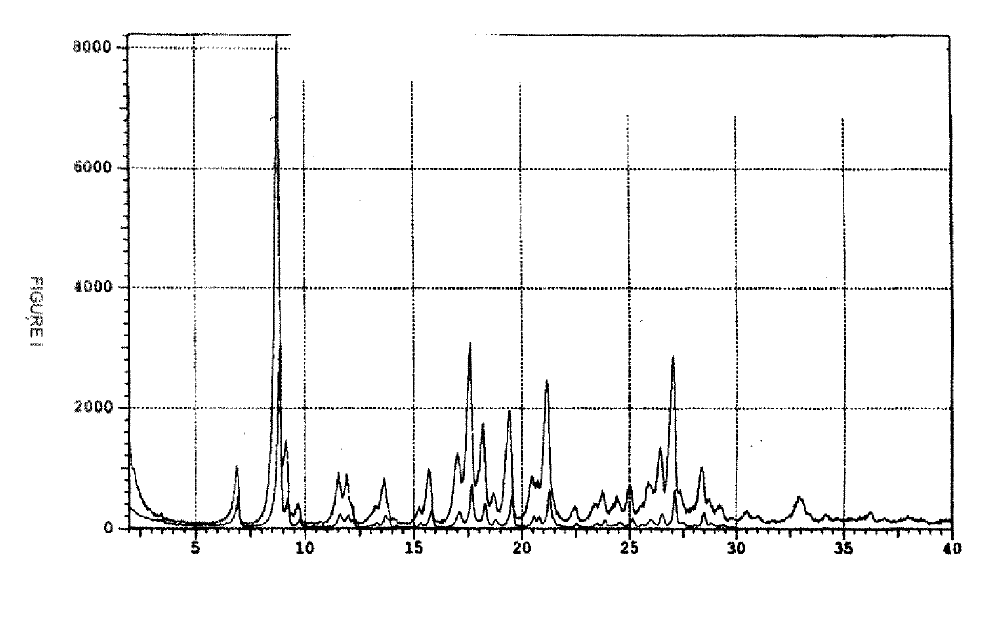

The inventors have found that the crystals of the compound of Formula I having desirably properties, Form 4 crystals, having an xray powder diffraction pattern shown in FIG. 1, precipitated from the reaction mixture resulting from the process shown in steps “a” and “b” of Scheme IIa display filter cake specific resistance of about 8.0×10¹¹ m/kg, as measured by measurement specific velocity of the slurry obtained from Scheme IIa, step “c(vi)” when filtered through a standard filter set up.

The inventors have surprisingly found that by seeding the reaction mixture with crystallites of the desired crystalline form of the compound of Formula I (Form 4), and subjecting the mixture to a temperature cycling regime in accordance with optional step “c” of the process according to Scheme IIa (above), crystals having lower filter cake specific resistance can be prepared, permitting facile scaleup of the process to a commercial scale and obviating long filtration time to isolated the compound of Formula I from the reaction mixture.

Although Scheme IIa utilizes n-propanol as a solvent and water as an anti-solvent, it will be appreciated that other alcohols, for example, but not limited to, alcohols having 6 carbon atoms or fewer, for example methanol, ethanol, and isopropanol, can also be employed in the process without departing from the scope of the present invention. Moreover, other solvents, when used in appropriate ratio with an anti-solvent, can be employed also, for example, but not limited to, acetone, acetonitrile, tetrahydrofuran, and N-methylpyrrolidine.

In general, the ratio of solvent to anti-solvent employed in the inventive process will be from about 5 vol % solvent:95 vol. % anti-solvent to about 98 vol. % solvent:2 vol. % anti-solvent. In some embodiments it is preferred to use solvent:anti-solvent in a volume ratio of 1:1, thus, a solvent system having a ratio of about 50 vol. % solvent:50 vol. % anti-solvent.

Without wanting to be bound by theory, it is believed that the crystalline Form 4 compound from a conventional crystallization step (single temperature excursion) has an l/d ratio which is very large, thus providing fragile crystal structure which is easily broken and “packs” efficiently during filtration, clogging the filters, and thus providing a mass having high filter cake specific resistance values. It is believed that the present process permits the l/d ratio of the crystals produced to be reduced, thereby permitting freer flowing filter cakes to be formed.

In some embodiments the present process comprises: (a) providing a solution comprising the compound of Formula I and a solvent/antisolvent mixture (crystallization medium) selected to afford a solution of the compound of Formula I when heated; (b) forming a solution of the compound of Formula I by heating the medium in the presence of the compound of Formula I; (c) cooling the solution thereby produced to a temperature proximal to the temperature at which solids begin to crystallize out of the solution (seeding temperature), (d) seeding the solution while held at the seeding temperature, thereby forming a mixture; (e) cooling the mixture in a controlled fashion to a temperature at which crystallization of the compound of Formula I proceeds (crystallization temperature) wherein the cooling rate is selected from a rate of from about 0.01° C./min. to about 5° C./min, thereby forming a slurry as cooling proceeds; and (f) cycling the temperature of the slurry thereby provided. In some embodiments it is preferred to cycle the temperature of the slurry by heating it to a temperature below the temperature of seeding employed in step “b” at a rate of from about 0.01° C./min. to about 5° C./min and cooling it to the crystallization temperature achieved in the step “c” at a cooling cycle rate of from about 0.01° C./min. to about 5° C./min, and repeating the temperature excursions at those heating and cooling rates until crystals of the desired size are obtained, thereby providing a mass of crystals capable of forming a filter cake having a filter cake specific resistance of less than 7.9×10¹¹ m/Kg when the precipitated crystals are isolated by filtration.

In some embodiments it is preferred to use n-propanol as the solvent in a 1:1 volumetric ratio with water as the anti-solvent in some embodiments using n-propanol, it is preferred to provide a solution of the compound of Formula I by acid work up of the reaction mixture produced in Scheme IIa, to remove added base, then concentrate the reaction mixture by distillation of volatiles, followed by the addition of n-propanol to the concentrate. In some embodiments the cycle of concentration by distillation with dilution by n-propanol is repeated until the resulting solution comprises primarily n-propanol. In some embodiments using the concentration/dilution method it is preferred to heat the resulting solution to 70° C. and add water while maintaining the temperature to provide a solution of the compound of Formula I in a crystallization medium. It will be appreciated that starting with an isolated solid form of the compound of Formula II, a solution can be provided by taking up an aliquot of the solid in n-propanol and adding water at the dissolution temperature, without departing from the scope of the invention. It will be appreciated that any scheme for providing a solution of the compound of Formula I in a crystallization medium will be useful in the process of the present invention.

In some embodiments using n-propanol as a solvent, after providing a solution of the compound of Formula I, it is preferred to seed the solution at a temperature of about 62° C. In some embodiments it is preferred to use a cooling rate of about 0.1° C./min. for the initial cooling cycle in step “c(v)” of Scheme IIa (after seeding the solution of Formula I to provide a mixture) until the mixture reaches a temperature of about 20° C. In some embodiments it is preferred to cycle the temperature by heating to a temperature of less than the seeding temperature and cooling the mixture a second time. In some embodiments the high temperature used in successive heating cycles is preferably 53° C. and the heating rate is preferably 0.5° C./min. In some embodiments, it is preferred to use successive cooling cycles to bring the mixture to a crystallization temperature of about 20° C. at a cooling rate preferably of 0.1° C./min. in some embodiments it is preferred to carry out at least 4 cycles of heating and cooling in step “c(v)” of Scheme IIa. In some embodiments it is preferred to carry out at least 8 cycles of heating and cooling in step “c(v)” of Scheme IIa.

In some embodiments, it is preferred to provide seed crystals by saving a portion of the crystals produced in one process for use as seed crystals in a subsequent process. With reference to Scheme IIa, in some embodiments using seed crystals prepared in accordance with the present process, it is preferred to carry out step “c(v)” using at least four heating and cooling cycles to provide the crystals.

For use in the process of the present invention, the preparation of the compound of Formula IV(i) [3-amino-2-hydroxy-benzamide] is described in U.S. Pat. No. 7,071,342 (the '342 patent), see for example, col. 23, lines 3 to 30.

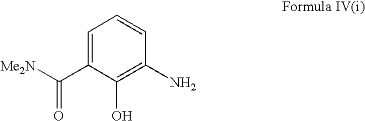

When reacted with hydrochloric acid, the compound of Formula IV(i) can be used to provide the amino-hydroxybenzamide salt compound of Formula 2B. In some embodiments it is preferred to produce the compound of Formula 28 from the compound of Formula IV(i) by treating a methyl-t-butyl ether/ethanol solution of the compound of Formula IV(i) with concentrated HCl. In some embodiments it is preferred to precipitate the salt product from an isopropanol/methyl-t-butyl ether solution by adding heptane as an antisolvent. It will be appreciated that other acid salts, produced using the same procedure can also be employed in the reaction of Scheme IIb. Suitable salts include, but are not limited to, hydrochloride, oxalate, p-tolylsulfonate, monobasic tartarate, and tartarate.

There follows non-limiting examples illustrative of the present invention but not limiting the present invention.

### EXAMPLES

Unless otherwise specified, all reagents are articles of commerce, food grade or pharmaceutical grade, and used as received.

### Example Ia

### In Situ Preparation of Dimethyl Squarate (2A2) and Reaction with Compound (2B) to Form Compound (2Ca)

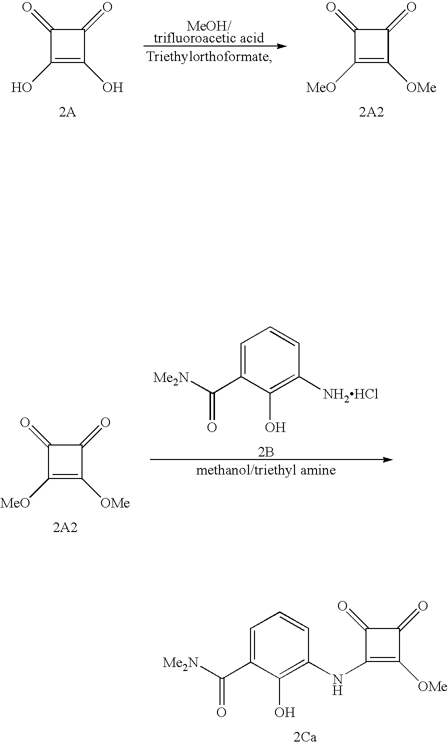

Into a 50 gallon glass reactor equipped with a thermocouple, N₂ inlet and feed tank was charged 9.5 kg of the compound of Formula 2A. The reactor was then charged with 65 liters dry methanol (Karl Fischer titration “KF” indicates water present at <0.1%) followed by 20 liters trimethylorthoformate and 0.2 kg trifluoroacetic acid. The reaction mixture was heated to reflux and maintained for about one hour. The reaction mixture was concentrated at one atmosphere until the internal temperature exceeded 70° C. The reaction mixture was maintained at reflux for about four hours then the temperature was adjusted to a temperature between 40° C. and 50° C. The reactor was charged with 26 liters dry methanol and the reaction mixture temperature was adjusted to about 20° C. to 30° C. The reactor was charged with 78 liters of dry methanol and the reaction mixture temperature was adjusted to a temperature between −5° C. and 5° C. The reactor was charged with 13.0 kg of the compound of Formula 2B. Triethylamine (TEA), 11.1 kg, was charged into the reactor over 4 hours while maintaining the batch at a temperature between −5 and 5° C. About one and a half hours after the start of the TEA charge, the reaction mixture was seeded with 130 grams of the compound 2C. After the addition of TEA was completed the reaction mixture was agitated for about 30 minutes maintaining the batch temperature between −5 and 5° C. Acetic acid, 12 liters was charged into the reactor while maintaining the batch at a temperature between −5 and 5° C. The reaction mixture was heated to a temperature between 60 and 70° C. and maintained in this temperature range for about 1 hour. After about 1 hour the temperature was adjusted to a temperature in the range of 25° C. to 35° C. and maintained at that temperature range for about 1 hour, then the temperature was readjusted to a temperature in the range of [−5° C.] to [+5° C.] over about 1 hour. The reaction mixture was filtered and the filter cake washed with 65 liters methanol. The solids collected were dried in a vacuum oven for about 24 hours with the oven temperature maintained at 60° C. to 70° C. Yield was 14.5 kg, about 81% based on the amount of the compound of Formula 2C employed.

¹HNMR (CD₃CN)

8.07 (1H, s); 7.56 (1H, d); 7.28 (1H, d); 6.99 (1H, t); 4.35 (3H, s); 3.10 (6H, s)

### Example Ib

### Preparation of the Compound of Formula (2Ca) from Commercial Dimethyl Squarate (2A2)

Charge 6.3 grams of the compound of Formula 2A1 (Aldrich, used as received) and 5.0 grams of the compound of Formula I to 250 ml round bottom flask equipped with a thermocouple, N₂ inlet and addition funnel. Charge 41 ml dry methanol (KF<0.1%), Adjust the batch to temperature between −5 and 5° C. Over about 5 hours, charge 4.9 ml (0.98×) triethylamine (TEA) to the batch while maintaining the batch at a temperature between −5 and 5° C. After the addition of TEA is complete, agitate the batch for about one hour at a temperature between [−5° C.] and [+5° C.]. Charge 2.8 ml acetic acid while maintaining the batch at a temperature between [−5° C.] and [+5° C.]. Adjust the batch volume to 63 ml by adding dry methanol. Heat the batch to reflux and maintain for about 15 minutes. Adjust the temperature to about [−5° C.] and [+5° C.] over about 1 hour. Filter the batch and wash the filter cake with 25 ml methanol. Dry the batch in a vacuum oven for at least 24 hours at 60 to 70° C. Yield 7.5 g, 88%.

### Example Ic

### Preparation of the Compound of Formula (2Ca) from Commercial Diethyl Squarate (2A3)

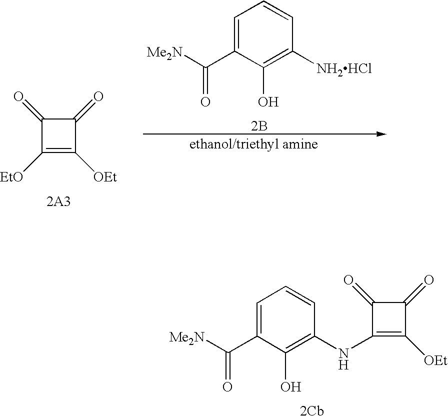

Charged 44.0 kg of the compound of Formula I, 225 kg dry ethanol and 41.8 kg of the compound of formula II to a 300 gallon glass lined reactor equipped with a thermocouple, N₂ inlet and feed bottle. Adjusted the batch to temperature between 0 and 10° C. Over about 1 hour, charged 17.1 kg triethylamine (TEA) to the batch while maintaining the batch at a temperature between 0° C. and 10° C. After the addition of TEA was complete, agitated the batch for about three hours at a temperature between 0° C. and 10° C. Over about 3 hours, charged additional 8.2 kg triethylamine (TEA) to the batch while maintaining the batch at a temperature between 0° C. and 10° C. After the addition of TEA was complete, agitated the batch for about three hours at a temperature between 0° C. and 10° C. Charged 19 liters acetic acid while maintaining the batch at a temperature between 0° C. and 10° C. Adjusted the batch volume to 440 liters by adding dry ethanol. Heated the batch to reflux and maintain for about 15 minutes. Adjusted the temperature to about 0° C. and 10° C. over about 2 hours. Filtered the batch and washed the filter cake with 220 liters 50% v/v ethanol in water. Dried the batch in a vacuum oven for at least 12 hours at 50 to 60° C. Yield 52 kg 88%.

¹HNMR (CD₃CN)

7.61 (1H, d); 7.28 (1H, t); 4.69 (2H, q); 3.10 (6H, s), 1.44 (3H, t).

### Example IIa

### Preparation of 2-Hydroxy-N,N-dimethyl-3-[[2-[[1(R)-(5-methyl-2-furanyl)propyl]amino]-3,4-dioxo-1-cyclobuten-1-yl]amino]benzamide Monohydrate (Form 4)

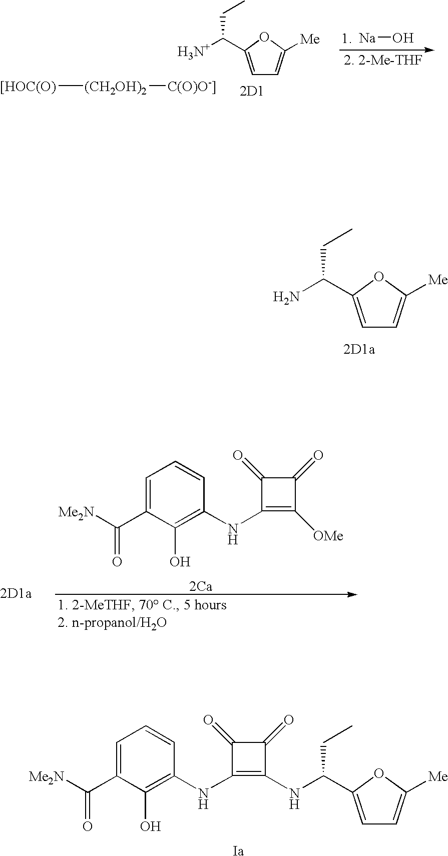

To a suspension of 10.1 g (2D1) (1.06 eq.) in 30 ml of water and 40 ml of 2-methyltetrahydrofuran was added 6.5 ml 32% of sodium hydroxide solution. The resulting aqueous layer was tested by pH paper. Additional small amount of caustic solution was added if pH was lower than 13. The organic was separated and the aqueous was extracted with 20 ml of 2-methyltetrahydrofuran. The combined organic layers was mixed with 10.0 g (1.0 eq.) of (2C) and the suspension was heated at 70° C. for 5 hours until the remaining starting material was below 1.0%. N-Propanol (50 ml) was added. The volume of the reaction mixture was reduced by distillation under partial vacuum to 40 ml (4×), followed by addition of 50 ml of n-propanol. The volume of the solution was reduced again under partial vacuum to 60 ml. The mixture was diluted to 90 ml with n-propanol and 0.3 ml of acetic acid was charged. The solution was then filtered. The filtrate was then diluted to 140 ml with n-propanol and the solution was heated to 70° C., Water (125 ml) was added while the batch temperature was maintained above 70° C. The solution was cooled to 62° C. and 200 mg (0.02×) seeds of the compound of Formula I (Form 4, previously prepared) were added. The mixture was stirred at 62° C. for 2 hours before it was cooled to 20° C. over about 5 hours. The suspension was then warmed up to 55° C. over 30 minutes before slowly cooling to 20° C. over 4 hours. The heating and cooling operation was repeated several times to grow crystals of the desired particle size. The suspension was finally cooled to 20° C. before filtration. The wet cake was washed with 80 ml solvent mixture of n-propanol and water (1:1). The cake was dried at 50° C. for 12 hours or until KF analysis showed the water content was below 47%, to give 11.5 g (85%) white needles, m.p. 83° C. XRD analysis showed the crystal form of the solids was form 4 monohydrate. ¹H NMR (DMSO-D6) δ, 0.91 (t, 3H, J=7.3), 1.84 (m, 1H), 1.94 (m, 1H), 2.25 (s, 3H), 2.92 (s, 6H), 5.13 (m, 1H), 6.01 (d, 1H, J=3.1), 6.25 (d, 1H, J=3.1), 6.85 (m, 2H), 7.78 (d, 1H, J=7.3), 8.65 (d, 1H, J=8.9), 9.29 (br, 1H), 9.99 (br, 1H). ¹³C NMR (DMSO-D6): 10.26, 13.32, 27.18, 52.78, 106.42, 107.52, 119.77, 120.76, 122.18, 124.42, 128.64, 143.25, 151.31, 152.06, 163.41, 168.27, 168.52, 180.17, 183.95, 184.71. Anal. calcd. for C₁₂H₂₅N₃O₆ (monohydrate 415.4): C, 60.71; H, 6.07; N, 10.11.

Found: C, 60.65; H, 5.93; N, 9.91.

### Example IIb

### Preparation of 2-Hydroxy-N,N-dimethyl-3-[[2-[[1(R)-(5-methyl-2-furanyl)propyl]amino]-3,4-dioxo-1-cyclobuten-1-yl]amino]benzamide Monohydrate (Form 4)

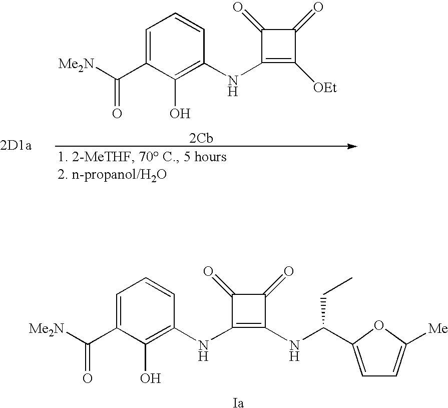

Following the same procedure used in Example IIa, 40.2 kg of 201 was treated with the base to make 2D1a, which was subsequently reacted with 39.8 kg of 2Cb (made previously from diethylsquarate), to give 43.8 kg (81%) of the title compound.

### Example III

### Preparation of 2-Hydroxy-N,N-dimethyl-3-amino-benzamide Salts

There follows four examples of the preparation of the hydrochloride, oxalate, p-tolylsulfonate, and tartarate salts of 3-amino-2-hydroxy-benzamide.

### Example III a

### Preparation of Compound 2B (HCl Salt of 3-amino-2-hydroxy-benzamide (Compound (IV(i)))

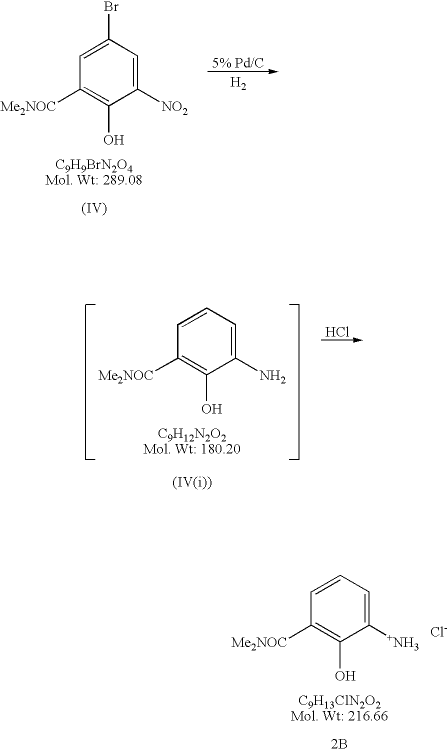

To a suspension of 10 g (34.6 mmol) of (IV) in a mixture of 21 ml of methyl t-butylether and 49 ml of ethanol was added 13.7 ml of KOEt (24%) in ethanol, followed by addition of 0.8 g of 5% Pd/C (50% wet). The mixture was then agitated under 120-150 psi hydrogen pressure for about 6 hours. Upon completion of the reaction, the batch was filtered through a Celite pad and the cake was washed with 80 ml of solvent mixture of methyl t-butylether and ethanol (1:1). The filtrate was treated with 3.7 ml of concentrated HCl solution. The batch was then concentrated under reduced pressure to about 50 ml. Isopropanol (100 ml) was added and the resulting solution was concentrated under vacuum to about 40 ml. Methyl t-butylether (50 ml) was added, followed by a slow addition of 110 ml of heptane. Finally, the mixture was cooled to 0° C. The solids were collected by filtration and the cake was washed with 20 ml solvent mixture of 1:1 methyl t-butylether/EtOH. The cake was dried at 60° C. for 10 hours in a vacuum oven, to give 7.24 g (96%) off-white solids of the compound of Formula 2B. ¹H NMR (DMSO-D6): 7.50 (d, 1H), 6.96 (dd, 1H), 7.17 (d. 1H), 2.9 (br, 6H), 10.2 (br, 4H), ¹³C NMR (DMSO-D6): 147.7, 121.4, 125.9, 120.6, 128.5, 127.1, 167.8.

### Example IIIb

### Preparation of 3-amino-2-hydroxy-benzamide Oxalate Salt (2B2)

Following the procedure described for preparing the HCl salt (28) in Preparative Example 1, 10 g (34.6 mmol) of compound (1V) was hydrogenated under the same condition and the filtered solution was treated with 3.3 g of oxalic acid. Following the same procedure as above resulted in 8.5 g (90%) off-white solids, ¹H NMR (DMSO-D6): 6.45 (m, 2H), 6.17 (dd, 1H), 2.70 (s, 6H). 5.5 (very broad, 4H).

### Example IIIc

### Preparation of 3-amino-2-hydroxy-benzamide p-Tolysulfonate Salt

Following the procedure described for preparing the HCl salt (2B) in Preparative Example 1, 10 g of compound (1V) was hydrogenated under the same condition and the filtrate was treated with 7.9 g (41.1 mmol) p-toluenesulfonic acid monohydrate. The resulting mixture was concentrated as above and the mixture after heptane addition was stirred over night at room temperature, to give 11.4 g (94%) off-white solids. ¹H NMR (DMSO-D6): 7.49 (d, 2H), 7.29 (d, 1H), 7.15 (m, 3H), 6.93 (dd, 1H), 2.90 (s, 6H), 2.31 (s, 3H).

### Example IIId

### Preparation of 3-amino-2-hydroxy-benzamide Tartarate Salt

Following the procedure described for preparing the HCl salt (28) in Preparative Example 1, 10 g of compound (1V) was hydrogenated under the same condition and the filtrate was treated with 5.47 g (36.5 mmol) of tartaric acid. Following the same procedure as described in 527123-PS preparation resulted in 9.1 g (80%) of off-white solids. ¹H NMR (DMSO-D6): 8.5 (br, 3H), 6.6 (dd, 2H), 6.38 (d, 1H), 4.26 (s, 2H), 3.6 (b, 2H), 2.96 (s, 6H).

### Example IV

### Preparation of 2-hydroxy-N,N-dimethyl-3-[[2-[[1(R)-[5-methyl-4-(1-methylethyl)-2-furanyl]propyl]-amino]-3,4-dioxo-1-cyclobuten-1-YL]amino]-benzamide (the compound of Formula II)

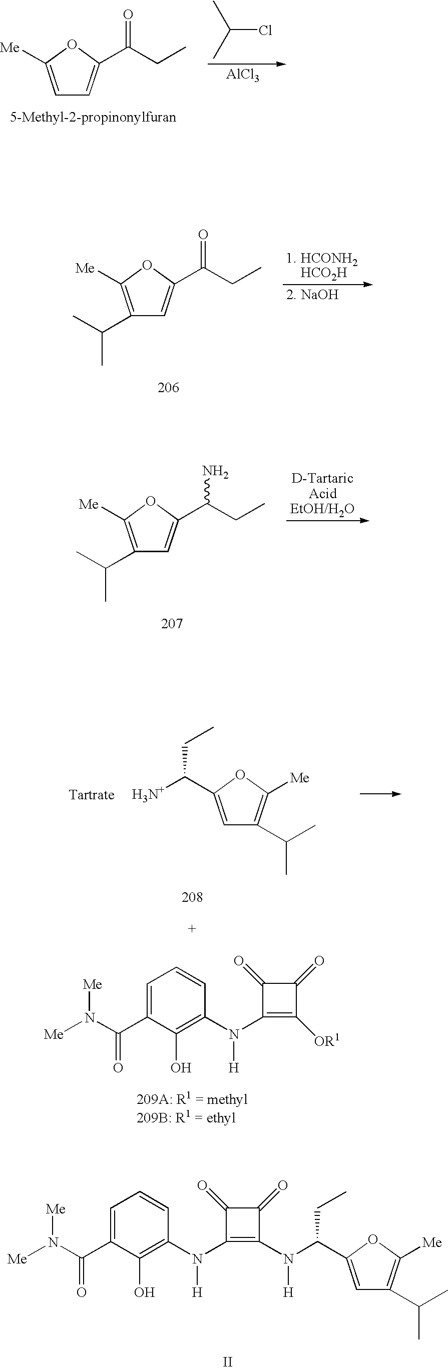

### Step 1: 1-(4-Isopropyl-5-methyl-2-furyl)propan-1-one (206)

Under nitrogen, 2-methyl-5-propionylfuran (100 g, 0.72 moles) was added dropwise at 0-30° C. to aluminium chloride (131 g, 0.96 moles). The resulting suspension was stirred for further 30 minutes at room temperature and then cooled to 0-5° C. Within one hour isopropyl chloride (76 g, 0.96 moles) was added dropwise at 0-10° C. and the mixture stirred until complete conversion was achieved (HPLC). The mixture was hydrolyzed on 2 L of water/ice. The pH was adjusted to 1 by addition of sodium hydroxide solution (60 mL) and the product was extracted into 500 mL TBME. The aqueous layer was separated and reextracted with 200 mL TBME. The combined organic layers were washed with 500 mL brine and evaporated to minimum volume. Yield: 132.5 g (102%) of a yellow-brown liquid. Assay (HPLC: YMC Pack Pro C18 150×4.6 mm, 5 μm: 220 nm; ACN/0.05% TFA:water/0.05% TFA 20:80 to 95:5 within 23 min): 60% pure by area. RT 17.2 min.

### Step 2: [1-(4-Isopropyl-5-methyl-2-furyl)propyl]amine (207)

Under nitrogen, a mixture of crude 1-(4-Isopropyl-5-methyl-2-furyl)propan-1-one (100 g), formamide (100 g, 2.22 moles) and formic acid (28.7 g, 0.61 moles) was heated to 140° C. for about two days until complete conversion to intermediate N-(1-(4-isopropyl-5-methylfuran-2-yl)propyl)formamide was achieved. The mixture was cooled to 20-25° C. and diluted with 400 mL methanol and 400 mL diisopropylether. Aqueous sodium hydroxide (1.2 kg, 25% in water) was added and the mixture was heated to reflux (55-60° C.) for about one day until complete conversion to [1-(4-Isopropyl-5-methyl-2-furyl)propyl]amine was achieved. The mixture was cooled down to 20-25° C. and the phases were separated. The organic layer was washed with 400 mL brine (5% in water). The combined aqueous layers were reextracted with 200 mL diisopropylether. The combined organic layers were evaporated to minimum volume. Yield: 94.6 g (45% abs (absolute), from 2-methyl-5-propionylfuran) of a yellow-brown liquid.

Assay (HPLC: YMC Pack Pro C18 150×4.6 mm, 5 μm; 220 nm; ACN/0.05% TFA:water/0.05% TFA 20:80 to 95:5 within 23 min): 48.5% pure vs. standard, RT 9.2 min.

### Step 3: (R)-1-(4-Isopropyl-5-methylfuran-2-yl)propan-1-amine (2S,3S)-2,3-dihydroxysuccinate (208)

Under nitrogen, crude [1-(4-isopropyl-5-methyl-2-furyl)propyl]amine (51 g, 135 mmol active) was dissolved in 204 mL dry ethanol at 60° C. 20% of a solution of D-(−)-tartaric acid (20.3 g, 135 mmol) in a mixture of 102 mL ethanol/water (15:1) was added at 55° C. The solution was seeded. The residual solution of tartaric acid was added within 10 minutes. The suspension was cooled to 20° C. and stirred at room temperature over night. The salt was filtered off and washed with dry ethanol until a colorless mother liquor was obtained. The product was dried in vacuum at 50° C. to constant weight. Yield: 16.9 g (38% abs.) of white crystals. Assay (HPLC: YMC Pack Pro C18 150×4.6 mm, 5 μm; 220 nm; ACN: 0.01M KH₂PO₄ pH=2.5 (H₃PO₄) 15:85 to 80:20 within 25 min): 95.8% by area, RT 8.8 min.

Optical Purity (HPLC: Chiralcel OD-R 250×4.6 mm; 226 nm; ACN: 0.5M NaClO₄ 40:60): dr 98:2, RT 116 min (R), 16.3 min (S). Wherein “dr” represents diastereomeric ratio.

### Step 4: 2-Hydroxy-3-[(2-{[(1R)-1-(4-isopropyl-5-methyl-2-furyl)propyl]amino}-3,4-dioxocyclobut-1-en-1-yl)amino]-N,N-dimethylbenzamide (Compound II)

Under nitrogen, (R)-1-(4-Isopropyl-5-methylfuran-2-yl)propan-1-amine (2S,3S)-2,3-dihydroxy-succinate (208) (2.0 g, 6 mmol) was suspended in 6 ml water and 8 mL 2-methyl tetrahydrofurane (MeTHF) at 20-25° C. 1.3 mL aqueous sodium hydroxide (30%) were added and the organic layer was separated after 5 minutes. The aqueous layer was extracted with 4 mL MeTHF. The combined organic layers were added to (209B) (1.74 g, 5.7 mmol) and 4 mL MeTHF were added. The mixture was heated to 65° C. for 4.5 hours and was then cooled to 20-25° C. After 16 hours at 20-25° C. the product crystallized and was isolated by filtration. The product was washed with MeTHF and dried in vacuum at 50° C. to constant weight. Yield: 1.25 g (47%) as off-white solid. Assay (NMR): 95% pure.

If one were to use compound (209A) in place of compound (209B) in Step 4 of Example IV, one would also obtain compound (II) using this same procedure.

There follows examples of the present invention controlled crystallization process yielding crystalline material having improved filter cake specific resistance. For each of these examples, the filter cake specific resistance was measured in accordance with the following procedure.

### Specific Cake Resistance Measurement Procedure V(i):

Into a pressure filter, the crystal slurry was filled. The filtration was then carried out under a constant pressure while the filtrate volume along with the filtration time was logged.

During the cake formation, the relationship between the filtrate volume and the filtration time can be described by the Tiller equation:

$$\frac{t}{V} = \frac{μα c}{2 {A}^{2} {g}_{c} Δ p} V + \frac{μ {R}_{m}}{{Ag}_{c} Δ p}$$

where: μ is the viscosity of filtrate, lb/ft-s or Pa·s; α is a specific cake resistance, ft, lb or ml/kg; c is the mass solid deposited in the filter per unit volume of filtrate, lb/ft³ or kg/m³: A is the filtration area, ft² or m²; g_{c} is Newton's law proportionality factor; p is the pressure, lbf/ft2 or atm; Rₘ is the filter-medium resistance, ft⁻¹ or m⁻¹.

The specific cake resistance a can be calculated from the slope of the linear plot of t/V vs. V.

### Example V

### Controlled Crystallization of the Compound of Formula I, 2-Hydroxy-N,N-dimethyl-3-[[2-[[1(R)-(5-methyl-2-furanyl)propyl]amino]-3,4-dioxo-1-cyclobuten-1-yl]amino]-benzamide Monohydrate (Form 4)

Following the general procedure of Example Iib, into a 2 L round bottom flask was placed 50.01 g of 2Da1, 50.0 g of 2Cb, 375 ml of n-propanol and 62.5 ml triethylamine. The batch was then agitated and heated up to 65° C. for 3 hours. After reaction completion, the batch was cooled down to 25° C. and filtered. The filtrate was collected and added 20 ml acetic acid. The batch volume was then adjusted to 540 ml with n-propanol.

### Example Va

### Recrystallization Procedure

The reaction mixture was split into two equal portions. The first portion was heated to 70° C. Purified water (183 mL) was slowly added to the first portion while maintaining the temperature at 70° C. The mixture was then cooled slowly to 62° C. and seeded with form 4 seeds. After holding at 62° C. for 1 hour, the batch was cooled down to 20° C. at a rate −0.1° C./min. The batch was then temperature cycled between 53° C. and 20° C. at a heating rate of 0.5° C./min and a cooling rate of −0.1° C./min for four times. The batch is then isolated and the wet cake washed with n-propanol/water mixture and dried under full vacuum at 50° C. for 14 hours. 20.16 g of dry product was obtained. PXRD results showed the dry product is crystalline form 4 material. The crystalline product thus produced was found to have a filter cake specific resistance of 6.4×10¹¹ m/Kg when the above-described filter resistance test was carried out.

### Example Vb

### Temperature Cycling Procedure

The second portion of the reaction mixture from Example Va was subjected to the same procedure as the first portion, however the batch was subjected to eight temperature cycles instead of four. A yield of 28.99 g dry product was obtained. The crystalline product thus produced was found to have a filter cake specific resistance of 2.5×10¹¹ m/Kg when the above-described filter resistance test was carried out.

### Example Vc

### Temperature Cycling Procedure

A portion (20.9 g) of the compound of Formula I obtained in accordance with the procedure of Example Vb was dissolved in 250.8 ml of n-propanol and 229.9 ml of purified water heated to 70° C. This solution was cooled to 60° C. and seeded with crystals of form 4 monohydrate. The seeded solution was held at 60° C. for 1 hour and cooled to 20° C. at a rate of 0.1° C./min. After the initial cooling period the batch temperature was cycled between 43° C. and 20° C. for 19 cycles to increase the particle size. The crystals produced were isolated and washed with n-propanol/water solvent mixture. The wet cake was dried under full vacuum at 50° C. for 4 hours. 17.8 g of dry product was obtained. The crystalline product thus produced was found to have a filter cake specific resistance of 1.99×10¹¹ m/Kg when the above-described filter resistance test was carried out.

The above description of the invention is intended to be illustrative and not limiting. Various changes or modifications in the embodiments described herein may occur to those skilled in the art. These changes can be made without departing from the scope or spirit of the invention

1. A process for providing a crystalline mass of the monohydrate Form 4 of the compound of Formula I affording filter cake specific resistance of less than 7.9×10¹¹ m/Kg, as measured in accordance with Specific Cake Resistance Measurement Procedure “V(i)” described herein, 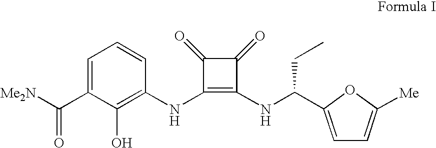
the process comprising:
(a) providing a solution of the compound of Formula I in a mixture of solvent and anti-solvent at the temperature of dissolution of the compound of Formula I afforded by the solvent/anti-solvent system selected;
(b) cooling the solution from step “a” to a temperature just above a temperature at which nucleation of the compound of Formula I commences in the solvent/antisolvent mixture selected for step “a” and seeding the batch with a solid crystalline form of the compound of Formula I monohydrate Form 4, thereby forming a mixture;
(c) cooling the mixture from step “b” to a temperature at which substantially all of the compound of Formula I dissolved in step “a” is crystallized into a slurry using a cooling rate of from about 0.01° C./min. to about 5° C./min; and
(d) cycling the temperature of the slurry from step “c” by heating it to a temperature below the temperature of seeding employed in step “b” at a rate of from about 0.01° C./min. to about 5° C./min and cooling the heated slurry at a rate of from about 0.01° C./min. to about 5° C./min to a temperature of about the crystallization temperature achieved in step “c” and repeating until crystals of the desired cross-section are obtained, thereby providing a filter cake specific resistance of less than 7.9×10¹¹ m/Kg when the precipitated crystals are isolated by filtration.

2. The process of claim 1 wherein the solvent is an alcohol having 6 carbon atoms or less, acetone, acetonitrile, tetrahydrofuran, N-methylpyrrolidine or mixtures of two or more thereof.

3. The process of claim 2, wherein the solvent is an alcohol having 3 carbon atoms or less.

4. The process of claim 3 wherein the solvent is n-propanol.

5. The process of any of claim 4 wherein the anti-solvent is water.

6. The process of claim 1 wherein the solution comprising the compound of Formula I used in step “a” is provided by a process comprising:
(a) forming the dialkyl-squarate of Formula A1 in situ by reacting (R³—O—)₃CH with squaric acid, 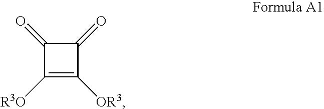
wherein R³ is selected from linear, branched, and cyclic alkyls of up to 10 carbon atoms;
(b) reacting the compound of Formula C, 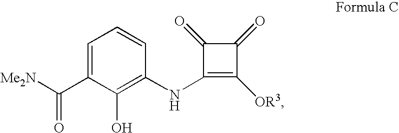
with a free base amino-furan compound of Formula D, 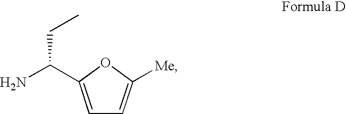
to provide the compound of Formula I, wherein R³ is defined above; and
(c) obtaining an n-propanol solution of the compound of Formula I by
(1) successive cycles of concentrating the reaction mixture formed in step “c” by distillation followed by the addition of an aliquot of n-propanol;
(2) adding an aliquot of acetic acid and n-propanol to the concentrate formed in step “1”;
(3) heating the solution formed in step “2”.

7. The process of claim 6 wherein the anti-solvent is water and is used in an amount to provide a solvent:anti-solvent ratio of from about 5 vol % n-propanol:95 vol % water to about 98 vol % n-propanol:2 vol % water.

8. The process of claim 7 wherein the volumetric ratio of solvent:anti-solvent employed in step “a” is 1:1 n-propanol:water.

9. The process of claim 8 wherein the dissolution temperature used in step “a” is about 70° C.

10. The process of claim 9 wherein the temperature just above nucleation to which the solution is cooled in step “b” is 62° C.

11. The process of claim 10 wherein the seeded mixture prepared in step “b” is cooled in step “c” to a temperature of about 20° C. at a rate of about 0.01° C./min.

12. The process of claim 11 wherein step “d” is carried out by employing a heating cycle that heats the mixture to about 53° C. at a rate of about 0.5° C./min and a cooling cycle that cools the mixture to a temperature of about 20° C./min. at a rate of 0.1° C./min.

13. The process of claim 12 wherein the crystals used to seed the solution in step “b” were previously prepared by the process of claim 1 using a step “d” having at least 4 heating/cooling cycles.

14. The process of claim 1 wherein, in step “d”, at least 4 heating/cooling cycles are preformed.

15. The process of claim 14 wherein at least 8 heating/cooling cycles are performed in step “d”.

16. A crystalline mass prepared by the process of claim 1, having an PXRD pattern of FIG. 1, and a filter cake specific resistance of less than 7.9×10¹¹ m/Kg.

17. The crystalline mass of claim 16 having a filter cake specific resistance of less than 6.4×10¹¹ m/Kg.

18. The crystalline mass of claim 16 having a filter cake specific resistance of less than 2.5×10¹¹ m/Kg.

19. The crystalline mass of claim 16 having a filter cake specific resistance of less than 2.5×10¹¹ m/Kg.

20. The process of claim 13 wherein, in step “d” at least 4 heating/cooling cycles are preformed.
